(19) United States
(12) Patent Application Publication
(10) Pub. No.: US 2012/0109217 A1
(43) Pub. Date: May 3, 2012

(54) ANTERIOR-TO-POSTERIOR TALUS-CALCANEUS SCREW INSERTION FOR ANKLE ARTHRODESIS NAIL

(76) Inventors: Christophe Perineau, Grenoble (FR); Jean-Emmanuel Cardon, Brignoud (FR)

(21) Appl. No.: 13/101,940

(22) Filed: May 5, 2011

Related U.S. Application Data
(60) Provisional application No. 61/332,091, filed on May 6, 2010.

(51) Int. Cl. A61B 17/86; A61B 17/56

(52) U.S. Cl. 606/301; 606/87

(57) ABSTRACT

Methods for anterior-to-posterior talus-calcaneus screw insertion through an ankle arthrodesis nail according to embodiments of the present invention include coupling a first jig to the nail, aligning a drill bit with a first aperture of the nail by inserting it through a second aperture in the first jig, drilling a first hole through a tibia aligned with the first aperture, fixing the nail to the tibia with a screw through the first hole into the first aperture, coupling a second jig to the first jig, aligning the drill bit with a third aperture in the nail by inserting the drill bit through a fourth aperture in the second jig, drilling a second hole through a talus and a calcaneus aligned with the third aperture; and fixing the nail to the talus and calcaneus by inserting a screw through the second hole into the third aperture in an anterior-to-posterior direction.

### CROSS-REFERENCE TO RELATED APPLICATIONS

This application claims the benefit of U.S. Provisional Patent Application Ser. No. 61/332,091, filed on May 6, 2010, which is incorporated herein by reference in its entirety for all purposes.

### TECHNICAL FIELD

Embodiments of the present invention relate generally to ankle arthrodesis, and more specifically to systems and methods for anterior-to-posterior talus-calcaneus screw insertion through an arthrodesis nail.

### BACKGROUND

Current talus-calcaneus screw insertion systems and methods involve a strictly posterior approach, which requires screws to be inserted posteriorly into the calcaneus, through an arthrodesis nail, and into the talus. Current jigs for such surgical procedures permit only posterior insertion of such screws.

### SUMMARY

A surgical apparatus according to embodiments of the present invention facilitates anterior-to-posterior insertion of screws through the talus, arthrodesis nail, and calcaneus, or through the calcaneus and the tibia. Such apparatus may include two jigs: a first jig configured for rigid attachment to an arthrodesis nail and/or to an intermediate piece extending from the arthrodesis nail, and a second jig configured for attachment to the first jig, according to embodiments of the present invention. When the first jig is coupled to the arthrodesis nail, and the second jig coupled to the first jig, holes in the second jig are aligned with holes in the arthrodesis nail, to permit drilling and/or insertion of fixation screws from anterior to posterior into the talus, through the nail, and through the calcaneus. According to embodiments of the present invention, the second jig may be removed from the first jig and flipped and/or reconfigured to permit a posterior insertion of the screws, using the same first jig and the same second jig.

A surgical method according to embodiments of the present invention includes attaching a first jig to an ankle arthrodesis nail, placing drill guides through holes in the first jig to drill holes through the tibia aligned with holes in the nail, inserting screws through the tibia holes and through the nail, coupling a second jig with the first jig, placing drill guides through holes in the second jig to drill holes through the talus and/or calcaneus aligned with holes in the nail, and inserting screws from anterior to posterior through the talus, through the holes in the nail, and into the calcaneus. According to embodiments of the present invention, the second jig is removed from the first jig, flipped and/or reoriented, and reattached to the first jig to permit a posterior approach using the same first and second jig.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a medio-anterior perspective view of an ankle with an arthrodesis nail and a tibial jig coupled to the arthrodesis nail, according to embodiments of the present invention.

FIG. 2 illustrates a perspective view of an arthrodesis nail, a nail connector, and a base tool, according to embodiments of the present invention.

FIG. 3 illustrates a medio-anterior perspective view of an ankle with an arthrodesis nail, a tibial jig coupled to the arthrodesis nail, and a talus-calcaneus jig coupled to the tibial jig in an anterior insertion position, according to embodiments of the present invention.

FIG. 4 illustrates a perspective view of a talus-calcaneus jig, according to embodiments of the present invention.

FIG. 5 illustrates a perspective view of the talus-calcaneus jig of FIG. 4 coupled to a tibial jig, according to embodiments of the present invention.

FIG. 6 illustrates another perspective view of a talus-calcaneus jig according to embodiments of the present invention.

FIG. 7 illustrates the medio-anterior perspective view of FIG. 3, with talus-calcaneus guides placed on the talus-calcaneus jig, according to embodiments of the present invention.

FIG. 8 illustrates a latero-posterior perspective view of the arthrodesis nail, tibial jig, and talus-calcaneus jig of FIGS. 1, 3, and 7 with the talus-calcaneus jig in a posterior insertion position, according to embodiments of the present invention.

FIG. 9 illustrates a front partial cross-sectional view of a right ankle joint with an ankle arthrodesis nail secured to the tibia with tibial screws and secured to the talus and calcaneus with screws applied anteriorly, according to embodiments of the present invention.

FIG. 10 illustrates a bottom partial cross sectional view of a right ankle joint with an ankle arthrodesis nail secured to the talus and calcaneus with screws applied anteriorly, according to embodiments of the present invention.

FIG. 11 illustrates a latero-anterior perspective view of the ankle joint of FIG. 9, according to embodiments of the present invention.

FIG. 12 illustrates a perspective view of an ankle arthrodesis jig system with a talus-calcaneus jig in a posterior-to-anterior insertion position, according to embodiments of the present invention.

FIG. 13 illustrates a perspective view of the ankle arthrodesis jig system of FIG. 12 with the talus-calcaneus jig in an anterior-to-posterior insertion position, according to embodiments of the present invention.

FIG. 14 illustrates a perspective view of an ankle with an alternative ankle arthrodesis system in a proximal fixation position, according to embodiments of the present invention.

FIG. 15 illustrates a perspective view of an ankle with the alternative ankle arthrodesis system of FIG. 14 in an anterior-to-posterior screw insertion position, according to embodiments of the present invention.

FIG. 16 illustrates a perspective view of an ankle with the alternative ankle arthrodesis system of FIGS. 14 and 15 in a posterior-to-anterior screw insertion position, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

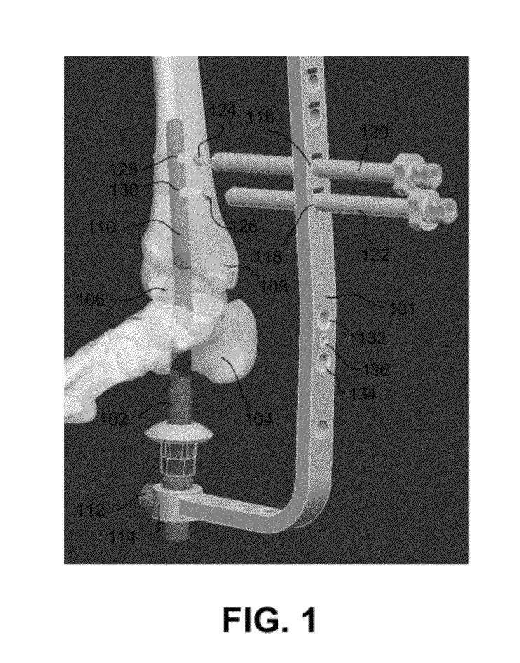

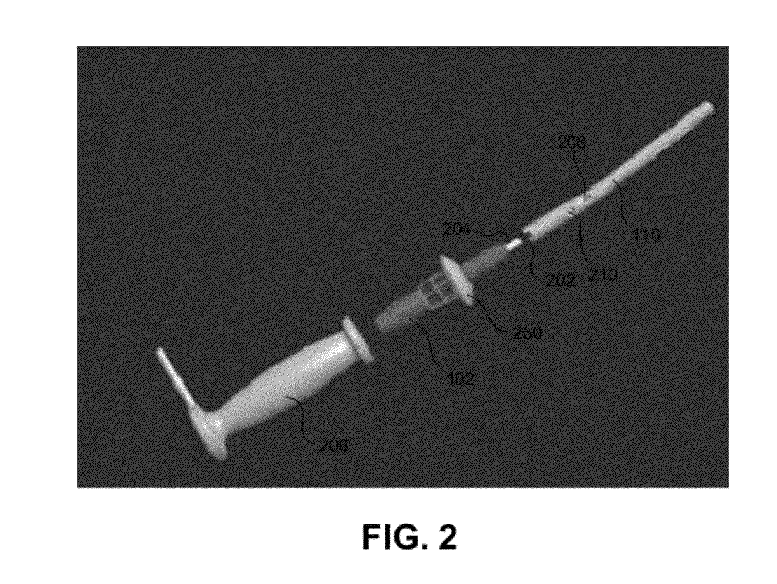

FIG. 1 illustrates a medio-anterior perspective view of an ankle with an arthrodesis nail 110 and a tibial jig 101 coupled to the arthrodesis nail 110, according to embodiments of the present invention. FIG. 2 illustrates a perspective view of arthrodesis nail 110, nail connector 102, and a base tool 206, according to embodiments of the present invention. The ankle joint includes several bones, including a tibia 108, a calcaneus 104, and a talus 106. According to embodiments of the present invention, the nail 110 has a slot, such as a female slot 202, that interfaces with a stem, such as a male stem 204, of the nail connector 102. According to some embodiments of the present invention, the connection between slot 202 and stem 204 prevents the nail connector 102 from rotating with respect to the nail 110; according to other embodiments, such connection prevents the nail connector 102 from moving at all with respect to the nail 110. A base tool 206 (see FIG. 2) may be removably coupled with the bottom of the nail connector 102; base tool 206 may assist the surgeon in placing and/or rotating the nail 110, according to embodiments of the present invention.

As illustrated in FIG. 1, once a hole has been drilled for the arthrodesis nail 110, and the nail 110 placed through the bottom of the ankle and up into the tibia 108, the tibia 108 is then secured to a proximal end of the nail 110, according to embodiments of the present invention. A tibial jig 101 may include a collar 114 on its bottom end, and the collar 114 may be configured to fit over and/or otherwise interface with the nail connector 102. A pin 112 may extend through collar 114 and into the nail connector 102 to couple the tibial jig 101 with the nail connector 102. Such a coupling creates a rigid connection of the arthrodesis nail 110 to the tibial jig 101, such that the tibial jig 101 cannot rotate and/or move with respect to the arthrodesis nail 110 after the pin 112 has secured the collar 114 to the nail connector 102, according to embodiments of the present invention. Based on the disclosure provided herein, one of ordinary skill in the art will appreciate the various other ways in which the tibial jig 101 may be coupled to the arthrodesis nail to prevent rotation and/or other movement of the tibial jig 101 with respect to the arthrodesis nail 110, according to embodiments of the present invention. The arthrodesis nail 110 may further include talus-calcaneus screw holes 208, 210, according to embodiments of the present invention.

The slot 202 and stem 204 interface may be configured to permit the nail connector 102 to couple with the nail 110 only at a predetermined rotational angle, and the nail connector 102 may include a slot or other indexing mechanism configured to interact with the pin 112 to permit the tibial jig 101 to couple with the nail connector 102 only at a predetermined angle, according to embodiments of the present invention. In this fashion, once the tibial jig 101 has been properly coupled to the arthrodesis nail 110, the surgeon will be able to rely on the fact that tibial guide holes 116, 118 on the tibial jig 101 will align with tibial screw holes 128, 130 on the arthrodesis nail 110, even though the surgeon will be unable to see the tibial screw holes 128, 130 after insertion of the arthrodesis nail 110, according to embodiments of the present invention.

In other words, when the tibial jig 101 is coupled to the nail 110 (e.g. via nail connector 102), a line drawn through tibial guide hole 116 intersects tibial screw hole 128 in the nail 110, and a line drawn through tibial guide hole 118 intersects tibial screw hole 130, according to embodiments of the present invention. According to embodiments of the present invention, an axial centerline of tibial guide hole 116 intersects tibial screw hole 128, and an axial centerline of tibial guide hole 118 intersects tibial screw hole 130.

Guides 120, 122 (e.g. trocars) may be inserted into tibial guide holes 116, 118. Guides 120, 122 may serve as drill guides, such that a hole drilled through guide 120 and tibia 108 will align with hole 128 in the nail 110, and a hole drilled through guide 122 and tibia 108 will align with hole 130 in the nail, according to embodiments of the present invention. Guides 120, 122 may also serve as screw insertion or driving guides, such that a tibial screw 124 inserted through guide 120 with a driving element will be aligned with and then driven into tibial screw hole 128, and a tibial screw 126 inserted through guide 122 will be aligned with and then driven into tibial screw hole 130, according to embodiments of the present invention. Tibial screws 124, 126 serve to fix the position of the arthrodesis nail 110 with respect to the tibia 108, according to embodiments of the present invention.

A flange 250 on the nail connector 102 permits compression applied to the plantar surface of the calcaneus 104 to compress the calcaneus 104 and talus 106 against the tibia 108; this may be done after fixation of the arthrodesis nail 110 to the tibia 108 via one or more screws 124, 126, according to embodiments of the present invention. According to some embodiments of the present invention, compression of the calcaneus 104 and/or talus 106 is achieved manually or in another way, and is not required for screw positioning.

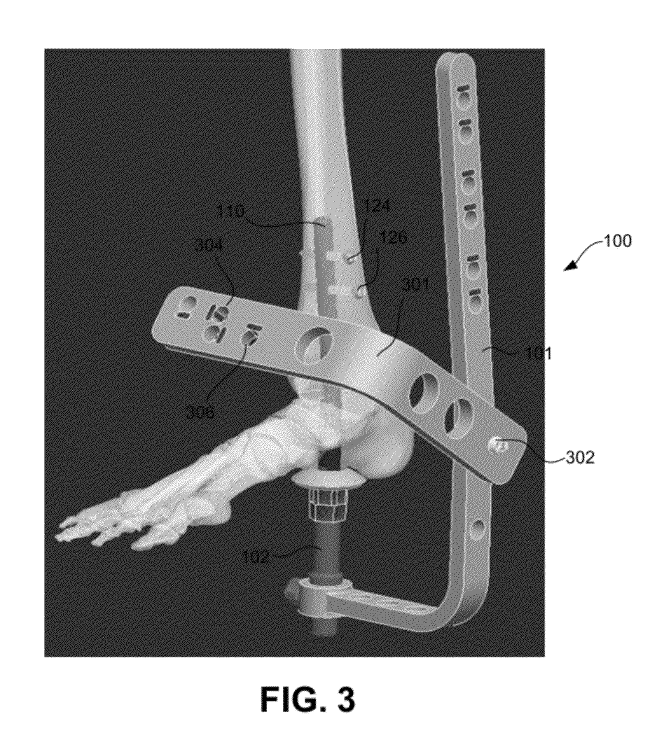

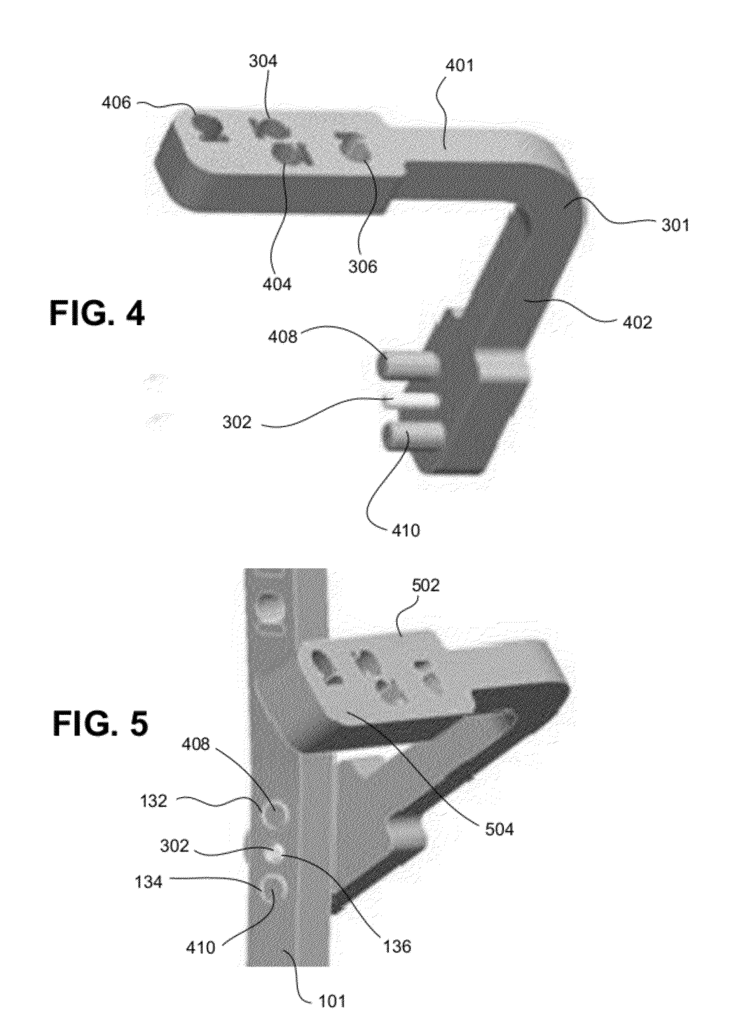

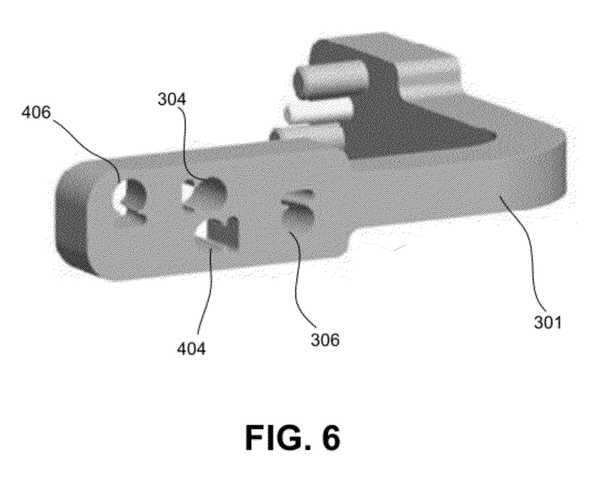

FIG. 3 illustrates a medio-anterior perspective view of an ankle with an arthrodesis nail 110, a tibial jig 101 coupled to the arthrodesis nail 110, and a talus-calcaneus jig 301 coupled to the tibial jig 101 in an anterior insertion position, according to embodiments of the present invention. FIG. 4 illustrates a perspective view of a talus-calcaneus jig 301, according to embodiments of the present invention. Once the arthrodesis nail 110 has been secured to the tibia 108, the nail 110 is then fixed to the talus 106 and calcaneus 104, according to embodiments of the present invention. FIG. 5 illustrates a perspective view of the talus-calcaneus jig 301 of FIG. 4 coupled to a tibial jig 101, according to embodiments of the present invention. FIG. 6 illustrates another perspective view of a talus-calcaneus jig 301 according to embodiments of the present invention. A talus-calcaneus jig 301 may be coupled to the tibial jig 101 for this purpose, according to embodiments of the present invention. The talus-calcaneus jig 301 may include one or more pegs 408, 410 rigidly affixed to the jig 301. Pegs 408, 410 may be configured for placement or insertion into holes 132, 134 in the tibial jig 101.

The pegs 408, 410 and holes 132, 134 are configured to position the talus-calcaneus jig 301 with respect to the tibial jig 101 (and thus with respect to the nail 110), such that when peg 408 is inserted into hole 132 and peg 410 is inserted into hole 134, a line drawn through hole 304 intersects hole 208 and a line drawn through hole 306 intersects hole 210, according to embodiments of the present invention. When peg 408 is inserted into hole 132, and peg 410 is inserted into hole 134, an axial centerline of hole 304 intersects hole 208 of the nail 110, and an axial centerline of hole 306 intersects hole 210 of nail, according to embodiments of the present invention. A screw 302 may be inserted through a hole in the talus-calcaneus jig 301 and into threaded engagement with a threaded hole 136 in the tibial jig 101, in order to rigidly couple the talus-calcaneus jig 301 to the tibial jig 101, according to embodiments of the present invention.

According to an alternative embodiment of the present invention, the pegs 408, 410 may be included by the tibial jig 301 and holes 132, 134 may be included by the talus-calcaneus jig 301. Based on the disclosure provided herein, one of ordinary skill in the art will appreciate the various ways in which the talus-calcaneus jig 301 may be coupled to the tibial jig 101 at a desired angle in order to permit alignment of the one or more holes 304, 306 with the one or more holes 208, 210 in the arthrodesis nail 110, according to embodiments of the present invention.

The talus-calcaneus jig 301 may include a guide arm 401 and a connection arm 402, and the guide arm 401 may be coupled with the connection arm at an approximate right angle (e.g. approximately ninety degrees), according to embodiments of the present invention.

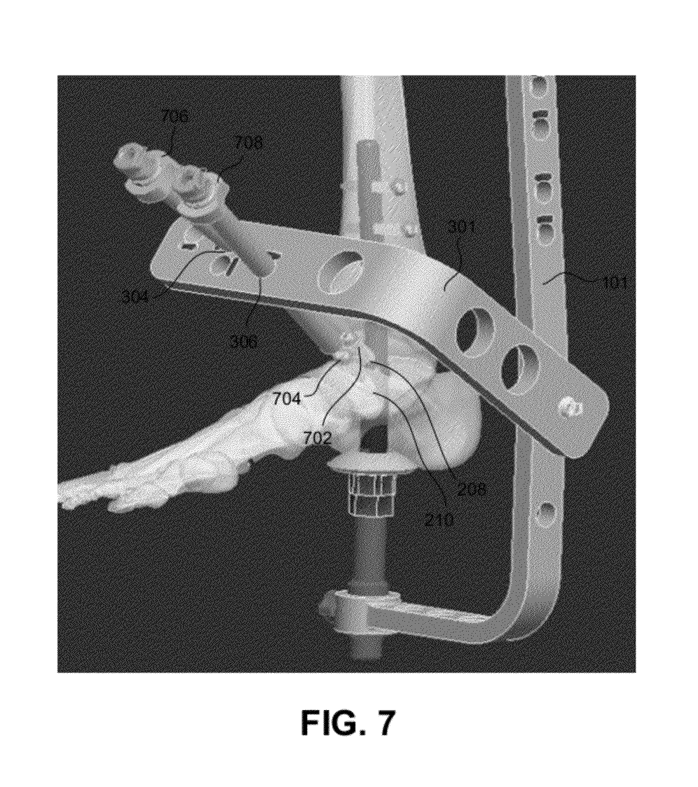

FIG. 7 illustrates the medio-anterior perspective view of FIG. 3, with talus-calcaneus guides 706, 708 placed on the talus-calcaneus jig 301, according to embodiments of the present invention. Guides 706, 708 (e.g. trocars) may be inserted into talus-calcaneus guide holes 306, 304. Guides 706, 708 may serve as drill guides, such that a hole drilled through guide 706 and talus 106 will align with hole 208 in the nail 110, and a hole drilled through guide 708 and talus 106 will align with hole 210 in the nail 110, according to embodiments of the present invention. Guides 706, 708 may also serve as screw insertion or driving guides, such that a talus-calcaneus screw 702 inserted through guide 706 with a driving element will be aligned with and then driven into talus-calcaneus screw hole 208, and a talus-calcaneus screw 704 inserted through guide 708 will be aligned with and then driven into talus-calcaneus screw hole 210, according to embodiments of the present invention. Talus-calcaneus screws 702, 704 serve to fix the position of the arthrodesis nail 110 with respect to the talus 106 and calcaneus 104, according to embodiments of the present invention.

Although holes 304, 306 are described as being aligned with holes 208, 210, the same is true for apertures passing through guides 706, 708, according to embodiments of the present invention. And although holes 116, 118 are described as being aligned with holes 128, 130, the same is true for apertures passing through guides 120, 122, according to embodiments of the present invention.

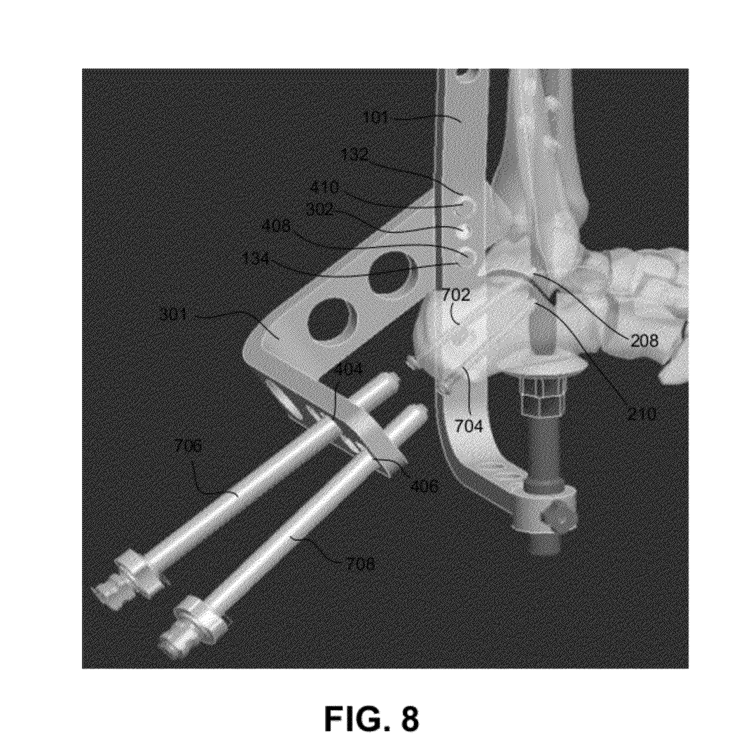

According to embodiments of the present invention, the talus-calcaneus jig 301 is removably coupled to the tibial jig 101, to permit the talus-calcaneus jig 301 to be switched between an anterior-to-posterior talus-calcaneus screw insertion position and a posterior-to-anterior talus-calcaneus screw insertion position. For example, screw 302 may be loosened and/or unthreaded from hole 136 to release the talus-calcaneus jig 301 from the tibial jig 101, by removing pegs 408, 410 from holes 132, 134. Then, peg 410 may be placed through hole 132 and peg 408 may be placed through hole 134, and talus-calcaneus jig 301 may again be attached to tibial jig 101 via screw 302, according to embodiments of the present invention. FIG. 8 illustrates a latero-posterior perspective view of the arthrodesis nail 110, tibial jig 101, and talus-calcaneus jig 301 of FIGS. 1, 3, and 7 with the talus-calcaneus jig 301 in a posterior-to-anterior insertion position, according to embodiments of the present invention.

FIG. 8 illustrates that the same talus-calcaneus jig 301 and tibial jig 101 combination can be used for both anterior-to-posterior and posterior-to-anterior arthrodesis screw insertion, according to embodiments of the present invention. As illustrated in FIGS. 4-6, the guide section 401 of the talus-calcaneus jig 301 may include multiple sets of holes. One set of holes 304, 306 may be configured to receive guides 706, 708 in order to guide drilling and screw 702, 704 insertion when the talus-calcaneus jig 301 is in the anterior-to-posterior screw insertion position of FIG. 7, according to embodiments of the present invention. The guide section 401 may include another set of holes 404, 406 configured to receive guides 706, 708 in order to guide drilling and screw 702, 704 insertion when talus-calcaneus jig 301 is in the posterior-to-anterior screw insertion position of FIG. 8, according to embodiments of the present invention. According to embodiments of the present invention, the talus-calcaneus jig 301 includes visual markings 502, 504 (see FIG. 5) indicating to the surgeon which holes are for anterior-to-posterior screw insertion and which holes are for posterior-to-anterior screw insertion. According to embodiments of the present invention, such visual markings 502, 504 include one or more of a color code, a word, a number, a pattern, a line, a notch or groove, and/or a protrusion. For example, the visual markings 502, 504 may include the letters “AP” connected by lines to the two holes corresponding to anterior-to-posterior screw insertion, and the letters “PA” connected by lines to the two holes corresponding to posterior-to-anterior screw insertion.

According to embodiments of the present invention, the talus-calcaneus jig is U-shaped and includes talus-calcaneus screw insertion holes on opposing ends, such that either anterior-to-posterior or posterior-to-anterior screw insertion may be accomplished using the same talus-calcaneus jig and without changing the position of the talus-calcaneus jig with respect to the tibial jig 101.

Embodiments of the present invention permit a surgeon to decide, during the surgical ankle arthrodesis procedure, whether to use an anterior-to-posterior screw insertion approach or a posterior-to-anterior screw insertion approach. Due to the ease of changing the position of the talus-calcaneus jig 301 with respect to the tibial jig 101, and due to the fact that the same tibial jig 301 piece of hardware may be used with the same tibial jig 101 for both approaches, a surgeon is provided with more flexibility both before and especially during the arthrodesis procedure. The surgeon may want to place the talus-calcaneus screws via an anterior-to-posterior method in order to limit the number of incisions, secure a bone graft in the talus with a screw, and/or in order to make the operation faster. Also, embodiments of the present invention permit a surgeon to decide between the anterior-to-posterior and posterior-to-anterior approaches based on an observation of the bone quality during surgery; for example, if a surgeon were initially planning an anterior-to-posterior insertion approach, and during surgery discovered the bone quality of the talus to be poor or not as suitable to accept the drilling and/or screw insertion, then the surgeon could simply change the configuration of the talus-calcaneus jig 301 without removing or adjusting the tibial jig 101 to implement a posterior-to-anterior approach.

When the talus-calcaneus jig 301 is in the posterior-to-anterior screw insertion position of FIG. 8, a line through hole 404 intersects hole 208 in the arthrodesis nail 110, and a line through hole 406 intersects hole 210 in the nail 110, according to embodiments of the present invention. According to embodiments of the present invention, an axial centerline of hole 404 intersects hole 208 and an axial centerline of hole 406 intersects hole 210. Guides 706, 708 (e.g. trocars) may be inserted into talus-calcaneus guide holes 404, 406. Guides 706, 708 may serve as drill guides, such that a hole drilled through guide 706 (e.g. a hole drilled using guide 706 as a guide) and calcaneus 104 will align with hole 208 in the nail 110, and a hole drilled through guide 708 and calcaneus 104 will align with hole 210 in the nail 110, according to embodiments of the present invention. Guides 706, 708 may also serve as screw insertion or driving guides, such that a talus-calcaneus screw 702 inserted through guide 706 with a driving element will be aligned with and then driven into talus-calcaneus screw hole 208, and a talus-calcaneus screw 704 inserted through guide 708 will be aligned with and then driven into talus-calcaneus screw hole 210, according to embodiments of the present invention. Talus-calcaneus screws 702, 704 serve to fix the position of the arthrodesis nail 110 with respect to the talus 106 and calcaneus 104, according to embodiments of the present invention.

Although a screw 302 is illustrated as providing the releasable coupling between the talus-calcaneus jig 301 and the tibial jig 101, one of ordinary skill in the art, based on the disclosure provided herein, will appreciate the numerous other ways in which the talus-calcaneus jig 301 may be releasably coupled with the tibial jig 101, according to embodiments of the present invention. For example, a quick-release and/or spring-loaded coupling mechanism may be employed, to permit the talus-calcaneus jig 301 to be pulled away from tibial jig 101 a sufficient distance to permit pegs 408, 410 to be removed from holes 132, 134, and then turned to the opposite configuration and released, permitting the pegs 408, 410 to re-engage opposite holes, according to embodiments of the present invention. Other types of quick-release and/or spring-loaded and/or slot-based mechanisms may be used, according to embodiments of the present invention.

Although guide members 120, 122, 706, 708 are described, the apertures 116, 118, 304, 306, 404, 406 may themselves be used to guide drill bits and/or screw insertion and/or screw driving, according to embodiments of the present invention.

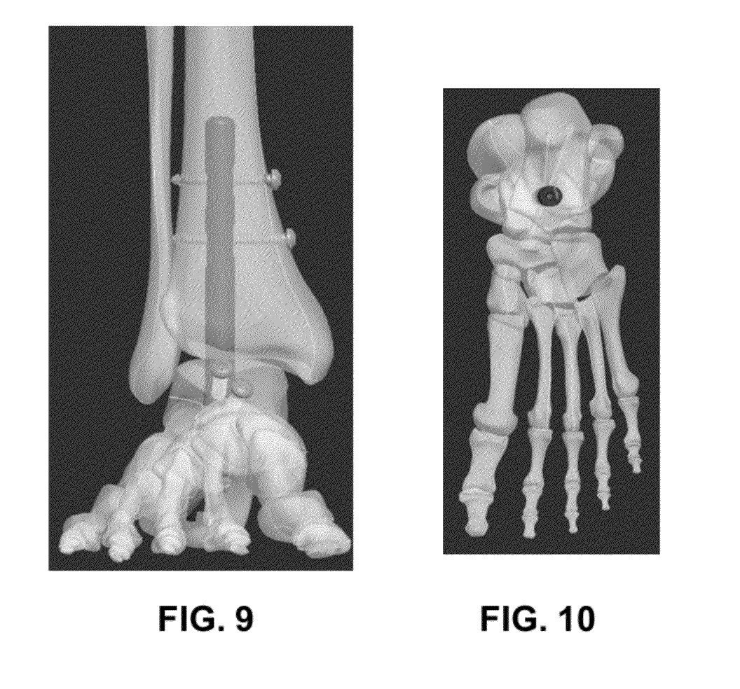

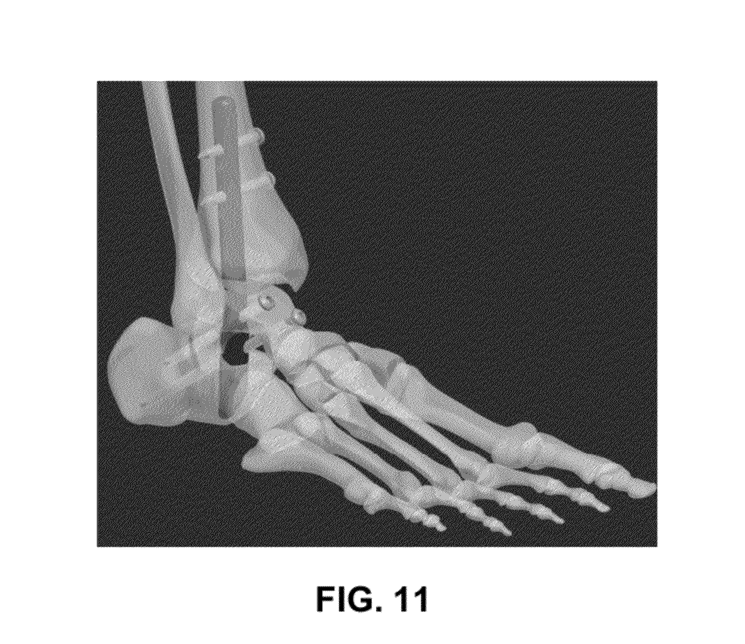

FIG. 9 illustrates a front partial cross-sectional view of a right ankle joint with an ankle arthrodesis nail secured to the tibia with tibial screws and secured to the talus and calcaneus with screws applied anteriorly, according to embodiments of the present invention. FIG. 10 illustrates a bottom partial cross sectional view of a right ankle joint with an ankle arthrodesis nail secured to the talus and calcaneus with screws applied anteriorly, according to embodiments of the present invention. FIG. 11 illustrates a latero-anterior perspective view of the ankle joint of FIG. 9, according to embodiments of the present invention.

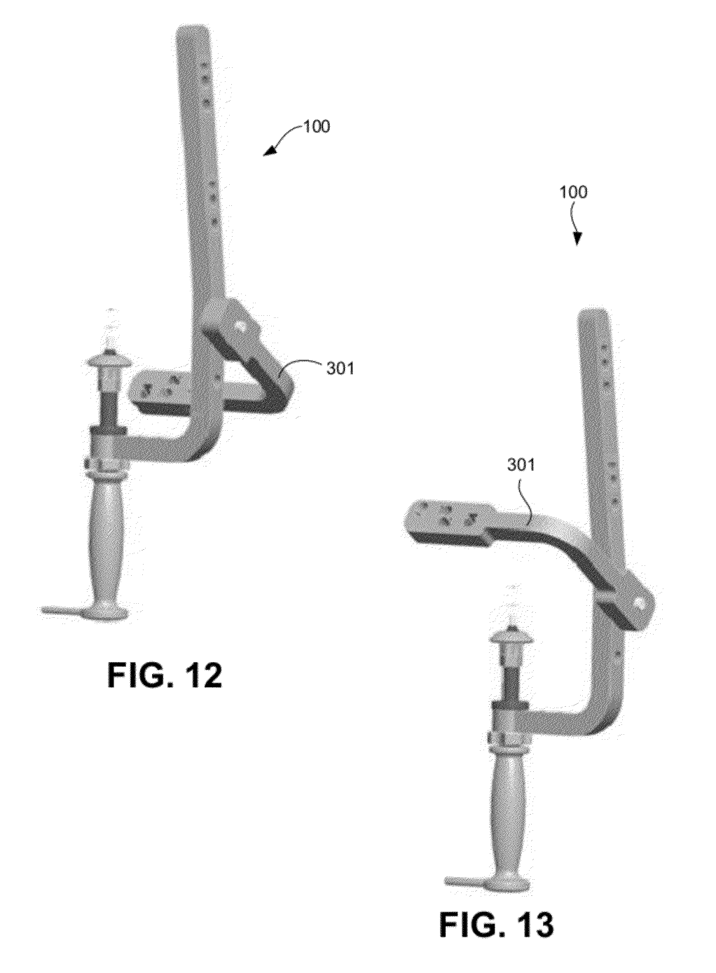

FIG. 12 illustrates a perspective view of an ankle arthrodesis jig system 100 with a talus-calcaneus jig 301 in a posterior-to-anterior insertion position, according to embodiments of the present invention. FIG. 13 illustrates a perspective view of the ankle arthrodesis jig system 100 of FIG. 12 with the talus-calcaneus jig 301 in an anterior-to-posterior insertion position, according to embodiments of the present invention.

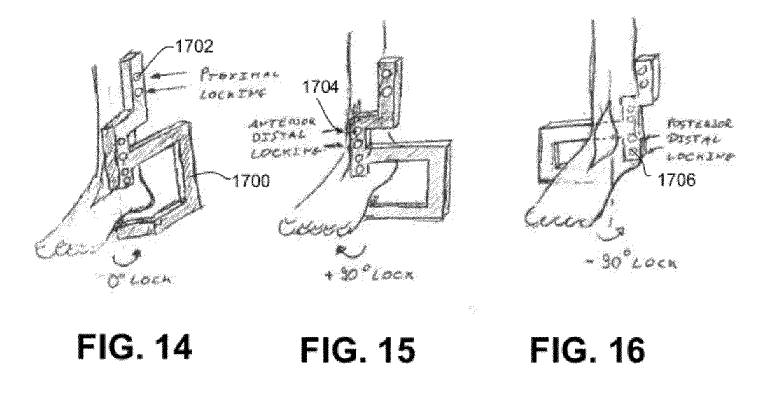

A jig according to some embodiments of the present invention does not include a talus-calcaneus element; instead, a single lateral jig may be coupled to the arthrodesis nail and rotated ninety degrees to the front and/or ninety degrees to the back, in order to insert screws in an anterior-to-posterior direction or in a posterior-to-anterior direction using the same single lateral jig. For example, FIGS. 14-16 illustrate such a jig 1700. Jig 1700 couples, for example rotatably couples, with the arthrodesis nail 110, according to embodiments of the present invention. Jig 1700 may be oriented in one of three discrete locked positions, according to embodiments of the present invention. In a “zero degree” lock position, as illustrated in FIG. 14, one or more tibial screw holes 1702 on jig 1700 are aligned with one or more holes 128, 130 of the nail 110 for fixation of the tibia with respect to the nail 110, according to embodiments of the present invention. In a “+90° lock position,” one or more anterior-to-posterior insertion holes 1704 are aligned with one or more holes 208, 210 in nail 110, as illustrated in FIG. 15, according to embodiments of the present invention. In a “−90° lock position,” one or more posterior-to-anterior insertion holes 1706 are aligned with one or more holes 208, 210 in nail 110, as illustrated in FIG. 16, according to embodiments of the present invention. Thus, the sets of one or more holes 1702, 1704, 1706 may be formed in a single rigid jig 1700 that is rotatable between two or more positions about the nail 110, according to embodiments of the present invention. For example, in one embodiment, jig 1700 rotates between the position of FIG. 14 and the position of FIG. 15, but not to the position of FIG. 16. In another embodiment, jig 1700 is rotatable between all three discrete locked positions of FIGS. 14-16. Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method for talus-calcaneus screw insertion through an ankle arthrodesis nail, the method comprising:
inserting a screw through a talus, a calcaneus, and the ankle arthrodesis nail in an anterior-to-posterior direction.

2. The method of claim 1, wherein inserting the screw through the arthrodesis nail comprises inserting the screw through a nail aperture in the arthrodesis nail.

3. The method of claim 2, further comprising coupling a jig to the arthrodesis nail such that a jig aperture on the jig substantially aligns with the nail aperture.

4. The method of claim 3, further comprising drilling a hole into the talus and the calcaneus using the jig aperture to guide the drilling.

5. The method of claim 4, wherein inserting the screw comprises inserting the screw through the jig aperture.

6. The method of claim 5, further comprising attaching the arthrodesis nail to a tibia.

7. The method of claim 6, wherein the nail aperture is a first nail aperture, wherein the screw is a first screw, and wherein attaching the arthrodesis nail to the tibia comprises inserting a second screw through the tibia and into a second nail aperture in the arthrodesis nail.

8. The method of claim 7, wherein the jig aperture is a first jig aperture, wherein the hole is a first hole, the jig further comprising a second jig aperture, the method further comprising drilling a second hole into the tibia using the second jig aperture to guide the drilling.

9. The method of claim 8, wherein inserting the second screw comprises inserting the second screw through the second jig aperture.

10. The method of claim 9, wherein the jig comprises a first jig element and a second jig element, wherein the first jig element comprises the first jig aperture, wherein the second jig element comprises the second jig aperture, and wherein the second jig element is releasably coupled to the first jig element, the method further comprising: releasing the second jig element with respect to the first jig element.

11. The method of claim 10, wherein the second jig element is configured to be releasably coupled to the first jig element in two fixed positions, wherein the two fixed positions include a posterior-to-anterior position and an anterior-to-posterior position, the method further comprising changing a position of the second jig element from a posterior-to-anterior position to an anterior-to-posterior position.

12. The method of claim 10, wherein releasing the second jig element with respect to the first jig element occurs during a surgical procedure.

13. A method for anterior-to-posterior talus-calcaneus screw insertion through an ankle arthrodesis nail, the method comprising:
coupling a first jig to the ankle arthrodesis nail;
aligning a drill bit with a first aperture in the ankle arthrodesis nail by inserting the drill bit through a second aperture in the first jig;
drilling a first hole through a tibia, the first hole aligned with the first aperture;
inserting a first screw through the first hole and into the first aperture to fix the ankle arthrodesis nail to the tibia;
coupling a second jig to the first jig;
aligning the drill bit with a third aperture in the ankle arthrodesis nail by inserting the drill bit through a fourth aperture in the second jig;
drilling a second hole through a talus and a calcaneus, the second hole aligned with the third aperture; and
inserting a second screw through the second hole in an anterior-to-posterior direction and into the third aperture to fix the ankle arthrodesis nail to the talus and the calcaneus.

14. A system for talus-calcaneus screw insertion through an ankle arthrodesis nail, the system comprising:
an ankle arthrodesis nail having a first aperture and a second aperture, the first and second apertures located on the ankle arthrodesis nail such that upon placement of the ankle arthrodesis nail in an ankle joint, the first aperture is within a tibia and the second aperture is within a talus or a calcaneus;
a first jig configured to be coupled to the arthrodesis nail, the first jig comprising a third aperture, wherein a line passing through the third aperture also passes through the first aperture when the first jig is coupled to the ankle arthrodesis nail;
a second jig configured to be removably coupled to the first jig in at least two orientations, the second jig comprising a fourth aperture and a fifth aperture, the at least two orientations comprising an anterior-to-posterior orientation in which a line passing through the fourth aperture also passes through the second aperture when the second jig is coupled to the first jig and the first jig is coupled to the arthrodesis nail, and a posterior-to-anterior orientation in which a line passing through the fifth aperture also passes through the second aperture when the second jig is coupled to the first jig and the first jig is coupled to the arthrodesis nail.

15. The system of claim 14, further comprising a marking on the second jig visually distinguishing the fourth aperture from the fifth aperture.

16. The system of claim 14, wherein the second jig comprises a first peg and a second peg, wherein the first jig comprises a first peg hole and a second peg hole, wherein the first peg is received by the first peg hole and the second peg is received by the second peg hole in the anterior-to-posterior orientation, and wherein the first peg is received by the second peg hole and the second peg is received by the first peg hole in the posterior-to-anterior orientation.

17. The system of claim 16, wherein the second jig further comprises a fastener hole, wherein the first jig comprises a threaded fastener aperture, and wherein the first jig is removably coupled to the second jig by a fastener extending through the fastener hole and into the threaded fastener aperture.

18. The system of claim 14, further comprising a driver or drill guide extending through the third aperture.

19. The system of claim 18, wherein the driver or drill guide is a first driver or drill guide, the system further comprising a second driver or drill guide extending through the fourth aperture.

20. The system of claim 14, wherein the ankle arthrodesis nail is a first ankle arthrodesis nail, the system further comprising a second ankle arthrodesis nail, the second ankle arthrodesis nail having a sixth aperture and a seventh aperture, the sixth and seventh apertures located on the second ankle arthrodesis nail such that upon placement of the second ankle arthrodesis nail in the ankle joint, the sixth aperture is within a tibia and the seventh aperture is within a talus or a calcaneus, wherein the first jig further comprises an eighth aperture, wherein a line passing through the eighth aperture also passes through the sixth aperture when the first jig is coupled to the second ankle arthrodesis nail.
